(19) United States
(12) Patent Application Publication
(10) Pub. No.: US 2021/0267250 A1
(43) Pub. Date: Sep. 2, 2021

(54) EFFECTS OF A SHORT-TERM FASTING MIMICKING DIET ON QUALITY OF LIFE AND TOLERANCE TO CHEMOTHERAPY IN PATIENTS WITH CANCER

(71) Applicant: Andreas Michalsen, Berlin (DE)

(72) Inventor: Andreas Michalsen, Berlin (DE)

(21) Appl. No.: 17/260,692

(22) PCT Filed: Oct. 24, 2018
(86) PCT No.: PCT/EP2018/079175

§ 371 Date: Jan. 15, 2021

Related U.S. Application Data
(60) Provisional application No. 62/699,808, filed on Jul. 18, 2018.

(51) Int. Cl. A23L 33/00; A61K 31/337; A61K 31/282; A61K 31/675; A61K 31/704; A61K 31/519; A61K 31/513; C07K 16/32; C07K 16/22; A61P 35/00; A23L 33/185; A23L 33/125; A23L 33/12

(52) U.S. Cl. CPC A23L 33/30; A23V 2002/00; A61K 31/282; A61K 31/675; A61K 31/704; A61K 31/519; A61K 31/513; C07K 16/32; C07K 16/22; A61P 35/00; A23L 33/40; A23L 33/185; A23L 33/125; A23L 33/12; A61K 31/337

(57) ABSTRACT

An anti-cancer agent for use in a method for the treatment of cancer, in particular gynecological cancer, in a patient, wherein the method comprises subjecting said patient to a plurality of therapy cycles, in particular chemotherapy cycles, with the anti-cancer agent, which can be a chemotherapeutic agent, and subjecting said patient to a fasting mimicking diet (FMD) for a period of 30-42 hours before each therapy cycle and 18-30 hours after each therapy cycle.

### TECHNICAL FIELD

The present invention concerns the technical field of the pharmaceutical and nutraceutical industry.

In particular, the invention relates to a method of reducing the side effects caused by cancer treatment with a defined dietetic regimen as well as to anti-tumor compounds for use in the treatment of cancer in association with a defined dietetic regimen.

### Prior Art

Experimentally, short-term fasting (STF) protects healthy cells against the adverse effects of chemotherapy while making tumor cells more vulnerable to it [1]. An increasing body of evidence from basic research points to beneficial effects of intermittent and periodic fasting in chronic disease [2, 3], [4], [5], [6, 7]. Fasting promotes pronounced changes in metabolic pathways and cellular processes such as stress response (hormesis), autophagy and decreases IGF-1 that affects other factors as Akt, Ras and mammalian target of rapamycin (mTOR) to downregulate cell growth and proliferation [8]. In primates and in rodents caloric restriction and intermittent fasting are associated with reduced cancer risk [9, 10]. Chemotherapy is a mainstay in the treatment of malignant tumors, but frequently limited by its toxicity. Recently it has been shown, that the effects of fasting on susceptibility to chemotherapy differ between normal cells and cancer cells, a phenomenon described as differential stress resistance (DSR) [11, 12]. Experimental data indicate that fasting states may promote the protection of normal cells, but not cancer cells during chemotherapy, as oncogenic pathways inhibit the stress resistance. In healthy cells pronounced metabolic and gene expression changes are induced by fasting, including upregulation of DNA repair pathways and antioxidants, partly mediated by the shut down of pathways such as IGF-1/Akt and mTOR [13].

As weight loss may negatively affect the prognosis of cancer patients with cancer, short-term fasting (STF) but particularly fasting mimicking diets (FMDs), are ideal solutions since they do not require a chronic reduction in calorie intake which would results in weight and lean body mass loss [14]. So far, experimental data consistently show that the combination of short-term fasting/FMD cycles with chemotherapy is effective in enhancing chemotherapeutic tolerability and efficacy and thus has high translational potential [1]. In a first case series on 10 patients with various types of cancer STF was found to be feasible and reduced severity of chemotherapy-induced side effects. [8].

In a recent randomized pilot-study, 13 women with HER2-negative breast cancer receiving neo-adjuvant chemotherapy were randomized to STF during chemotherapy or to eat a common healthy diet. Fasting reduced the hematological toxicity of chemotherapy and induced a faster recovery of DNA damage [12].

In a recent pilot study De Groot et al. investigated a 48 h zero-calorie fasting during TAC chemotherapy in 13 women with confirmed HER-2-negative stage II and III breast cancer. Beside metabolic, endocrine and hematologic parameters, DNA damage in PBMCs was also assessed and side effects were evaluated according to the Common Terminology Criteria for Adverse events (CTCAE). 2 patients withdrew from fasting after the third chemotherapy because of clinical deterioration not related to fasting. Fasting was safe and had beneficial effects on hematologic toxicity and possibly on DNA damage in healthy cells (lymphocytes and myeloid cells) [19].

In a further report, a recent uncontrolled dose-escalation and feasibility study, Dorff et al. investigated 20 patients with three different fasting periods (24, 48 and 72 h). 16 of the patients were compliant with the fasting regimen (<200 kcal/day). Fasting was found to be safe and feasible for the cancer patients. There was also some preliminary evidence of reduced DNA-damage evident in host leukocytes after chemotherapy exposure for subjects who fasted for 72 h compared to 24 h in this study [20].

The Applicants addressed their research activity to the assessment of the effect of a FMD on quality of life (QOL) in patients with gynecological cancer undergoing chemotherapy cycles. Based on the experimental evidence, the Applicants hypothesized that fasting increases QOL and reduces fatigue during chemotherapy compared to standard nutrition [15]. By means of an explorative cross-over design the Applicants compared QOL, general well-being and fatigue across all fasted chemotherapy cycles versus all chemotherapy cycles with normocaloric diet and found that application of determined periods of FMD before and after chemotherapy cycles actually brings about a significant increase of QOL and a significant reduction of fatigue. This is reported in the article of Baursfeld et al. “The Effects of Short-Term Fasting on Quality of Life and Tolerance to Chemotherapy in Patients with Breast and Ovarian Cancer: a Randomized Cross-Over Pilot Study”, (BMC Cancer 2018 18:476), published on Apr. 27, 2018 and representing inventor's own work.

The present invention is the result of the above research activity.

### SUMMARY OF THE INVENTION

In an aspect thereof, the present invention relates to an anti-cancer agent for use in a method for the treatment of cancer in a patient, wherein the method comprises subjecting said patient to a plurality of therapy cycles with said anti-cancer agent and subjecting said patient to a fasting mimicking diet (FMD) for a period of 30-42 hours before each therapy cycle and 18-30 hours after each therapy cycle.

In another aspect, the present invention relates to a method of increasing the Quality-Of-Life (QOL) and reducing the fatigue of a human patient with a cancer subjected to therapy cycles with an anti-cancer agent, which method comprises subjecting said patient to a plurality of therapy cycles with said anti-cancer agent and subjecting said patient to a fasting mimicking diet (FMD) for a period of 30-42 hours before each therapy cycle and 18-30 hours after each therapy cycle.

Preferably said plurality of therapy cycles consists of 4-6 therapy cycles.

Preferably, the above-mentioned period of FMD is of 33-39 hours, more preferably 36 hours, before each therapy cycle, and of 21-27 hours, more preferably 24 hours, after each therapy cycle.

The anti-cancer agent is preferably a chemotherapeutic agent, but it can also be any other agent that is used for the treatment of cancer, including cytotoxic agents and antimetabolites.

The chemotherapeutic agent is preferably selected from a group comprising DNA alkylating agents, taxanes, platinum agents, cyclophosphamide, anthracyclines, topoisomerase inhibitors, kinase inhibitors, nucleotide analogs, retinoids, peptide antibiotic, Vinca alkaloids, epothilones, histone deacetylase inhibitors, antibodies; advantageously, the chemotherapeutic is selected from the group comprising docetaxel, paclitaxel, carboplatin, cyclophosphamide, epirubicin, doxorubicin, methotrexate, fluorouracil, bevacizumab, pertuzumab and trastuzumab.

As used herein, “cancer” refers to a disease or disorder characterized by uncontrolled division of cells and the ability of these cells to spread, either by direct growth into adjacent tissue through invasion, or by implantation into distant sites by metastasis. Examples of cancers include, but are not limited to, primary cancer, metastatic cancer, carcinoma, lymphoma, leukemia, sarcoma, mesothelioma, glioma, germinoma, choriocarcinoma, prostate cancer, lung cancer, breast cancer, colorectal cancer, gastrointestinal cancer, bladder cancer, pancreatic cancer, endometrial cancer, ovarian cancer, melanoma, brain cancer, testicular cancer, kidney cancer, skin cancer, thyroid cancer, head and neck cancer, liver cancer, esophageal cancer, gastric cancer, intestinal cancer, colon cancer, rectal cancer, myeloma, neuroblastoma, pheochromocytoma, and retinoblastoma.

Preferably, the cancer is gynecological cancer, in particular breast cancer and ovarian cancer, but FMD is expected to work in protecting patients with any kinds of cancer against the side effects of cancer therapy with anti-cancer agents, including but not limited to the side effects of chemotherapy.

By FMD it is hereby meant a specific food composition providing a daily caloric intake reduced by 10-95%, preferably by 50-90%, more preferably by 85-90%, with respect to the regular caloric intake, including total starvation.

The above-mentioned reduced caloric intake preferably corresponds to a caloric intake of 100-350 kcal/day.

The subject's regular caloric intake is the number of kcal that the subject consumes to maintain his/her weight. The subject's normal caloric intake may be estimated by interviewing the subject or by consideration of a subject's weight. As a rough guide, subject's normal caloric intake is on average 2600 kcal/day for men and 1850 kcal/day for women.

The reduced caloric intake is obtained by means of a Fasting Mimicking Diet with reduced caloric, sugar and/or protein content but containing all necessary micronutrients to prevent malnutrition.

Preferably, when the daily caloric intake is reduced by 10-95%, the patient is fed with foods with a high content of monounsaturated and polyunsaturated fats and a reduced content of proteins and sugars (40% of calories coming from fat). This because a diet based on such foods has differential effects on normal and cancer cells that are similar to those caused by starvation.

In another aspect, the present invention relates to a pharmaceutical composition comprising an anti-cancer agent as defined above, preferably a chemotherapeutic agent, and a pharmaceutically acceptable carrier for use in a method as defined above.

In a further aspect, the present invention relates to an improved method of treating a human patient with cancer, comprising a step of subjecting said patient to a plurality of therapy cycles with an anti-cancer agent, the improvement consisting in subjecting the patient to a FMD for a period of 30-42 hours before each therapy cycle and 18-30 hours after each therapy cycle, thus obtaining an increase of the patient's Quality-Of-Life (QOL) and a fatigue reduction.

Preferably said plurality of therapy cycles consists of 4-6 therapy cycles.

In this method, the period of FMD is generally of 33-39 hours, preferably 36 hours, before each therapy cycle, and of 21-27 hours, preferably 24 hours, after each therapy cycle.

The anti-cancer agent to be used in this method is one of the agents mentioned here before and the calories provided by the FMD are reduced by 10-95%, preferably by 50-90%, which preferably corresponds to a caloric intake of 100-350 kcal/day.

The above-mentioned therapy cycles are preferably chemotherapy cycles.

The compounds and compositions according to the invention may be administered with any available and efficient delivery system, comprising, but not limited to, oral, buccal, parenteral, inhalatory routes, topical application, by injection, by transdermic or rectal route (for ex. by means of suppositories) in dosage unit formulations containing conventional, pharmaceutically acceptable and non-toxic carriers, adjuvants and vehicles. The administration by parenteral route comprises subcutaneous, intravenous, intramuscular, intrasternal injection or infusion techniques.

The solid dosage forms for the administration by oral route comprise, for example, capsules, tablets, powders, granules and gels. In such solid dosage forms, the active compound may be mixed with at least one inert diluent such as, for example, sucrose, lactose or starch. These dosage forms normally also comprise additional substances different from the inert diluents, such as, for example, lubricating agents like magnesium stearate.

The injectable preparations, for example aqueous or oily sterile injectable solutions or suspensions, may be formulated according to the known technique and by optionally using appropriate dispersing, wetting and/or suspending agents.

The pharmaceutical preparations according to the present invention may be produced by using conventional pharmaceutical techniques, as described in the various pharmacopoeias or handbooks of the field such as, for example, “Remington's Pharmaceutical Sciences Handbook”, Mack Publishing, New York, 18th Ed., 1990.

The average daily dosage of the compounds according to the present invention depends on many factors, such as, for example, the seriousness of the disease and the conditions of the patient (age, weight, sex): The dose may generally vary from 1 mg to 1500 mg per day of compound according to the invention, optionally divided into more administrations.

FMD with a period of 60 h did not induce weight loss and was associated with only minor adverse effects that were rated as not meaningful by the patients and did not interfere with daily activities.

In general, FMD led to a better tolerance to therapy, in particular chemotherapy, with less compromised QOL and reduced fatigue within the 8 days after the therapy.

Examples of FMD protocols that can be used in the present invention are found in U.S. patent application Ser. No. 12/430,058 and U.S. Ser. No. 13/488,590.

In one embodiment, the fasting mimicking diet provides the subject with a protein amount less than or equal to 20 g/day.

If carbohydrates are present in the fasting mimicking diet, they provide no more than half of the calories provided by the aforementioned diet.

Other examples of FMD can be found in the WO 2014/066426 and WO 2014/127000 applications.

Preferably, the fasting mimicking diet comprises proteins in an amount that is less than 15% of the total calories provided by the fasting mimicking diet.

Preferably, the fasting mimicking diet comprises sugars in an amount that is less than 5% of the total calories provided by the fasting mimicking diet

Preferably, the fasting mimicking diet comprises at least 60% calories from fatty acids, 2-5% calories from glycerol and up to 5% calories from plant-based proteins and a maximum of 35% calories from carbohydrates.

Preferably, the fasting mimicking diet comprises complex carbohydrates from plant sources, which preferably comprise mushrooms, kale, broccoli, tomatoes, spinach, carrots, pumpkin, etc.

Preferably at least 50% of the calories from fatty acids are from olive oil, coconut oil and tree nuts. The latter preferably comprise walnuts, macadamia nuts and/or almonds.

The present invention will be further described with reference to the appended drawings and to certain embodiments, which are provided here below by way of illustration and not of limitation.

### BRIEF DESCRIPTION OF THE DRAWINGS

FIG. 1 is a flow chart of the experimental study.

FIG. 2a illustrates the pre-test values at cycles (C1-C6) in FACIT-F(FS), FACIT-F, FACIT-F TOI, FACT-G, and Total FACIT-F, Group A, Day 0

FIG. 2b shows the pre-test values at cycles (C1-C6) FACIT-F(FS), FACIT-F, FACIT-F TOI, FACT-G, and Total FACIT-F Group B, Day 0

FIG. 3 shows: FACT-G Forest plot of mean difference a) Group A (pre-test-day 8, cycles c1-c3=fasting, i.e. FMD) b) Group B (pre-test-day 8 (cycles c1-c3=normocaloric) c) Group B (pre-test(c4)-day 8, cycles c4-c6=fasting, i.e. FMD) and d) Group A (pre-test(c4)-day 8, cycles c4-c6=normocaloric)

### DETAILED DESCRIPTION

The Applicants designed their study as a randomized, individually controlled cross-over trial. The study design was chosen on the background of the anticipated heterogeneity of the study population and therapy protocols. All study participants gave their written informed consent. The study protocol was reviewed and approved by the Ethics Committee of the Charite-University Medical Center, Berlin (EA4/088/13). Patients were enrolled between November 2013 and March 2015; interventions and follow-up were completed by August, 2015. Study procedures and data collection were carried out at the outpatient department of the Charité-University Berlin at Immanuel Krankenhaus Berlin.

### Study Procedures

Patients were referred by three gynecological hospital departments, two centers for breast cancer care and the Charite European Center for ovarian cancer. Potential participants were screened for eligibility during an appointment at the study center, and eligible candidates were scheduled for an enrollment appointment. Each eligible participant was randomly assigned to two different sequences of nutrition during the scheduled chemotherapy. Group A was randomized to a FMD of 60 h during the first three of scheduled 6 chemotherapies (36 h before to 24 h after the chemotherapy) followed by normocaloric nutrition during the following 3 chemotherapies. Group B was allocated to a vice versa sequence of nutrition. Between the chemotherapy cycles all patients were advised to follow their common diet. All patients received an approximate 1 h counselling by dieticians experienced in fasting treatments.

All measurements were performed baseline and 8 days after each chemotherapy cycle. Subjects height/body weight were measured following a standardized protocol. For assessment of QOL, general well-being and fatigue validated inventories were used. Adverse effects were assessed by two 2 interviews during and at the end of FMD and by means of a diary.

### Patients

All women had confirmed diagnosis of breast cancer or ovarian cancer and a scheduled chemotherapy. Eligibility criteria included age 18 years; BMI≥19 kg/m2; WHO performance status 0-2; anticipated life expectancy of >3 months; Exclusion criteria included: Type-1 diabetes or intensified insulin treatment; myocardial infarction, stroke or pulmonary embolism within the last 3 months; unstable heart disease; renal failure, history of eating disorder; dementia, psychosis, impaired physical mobility.

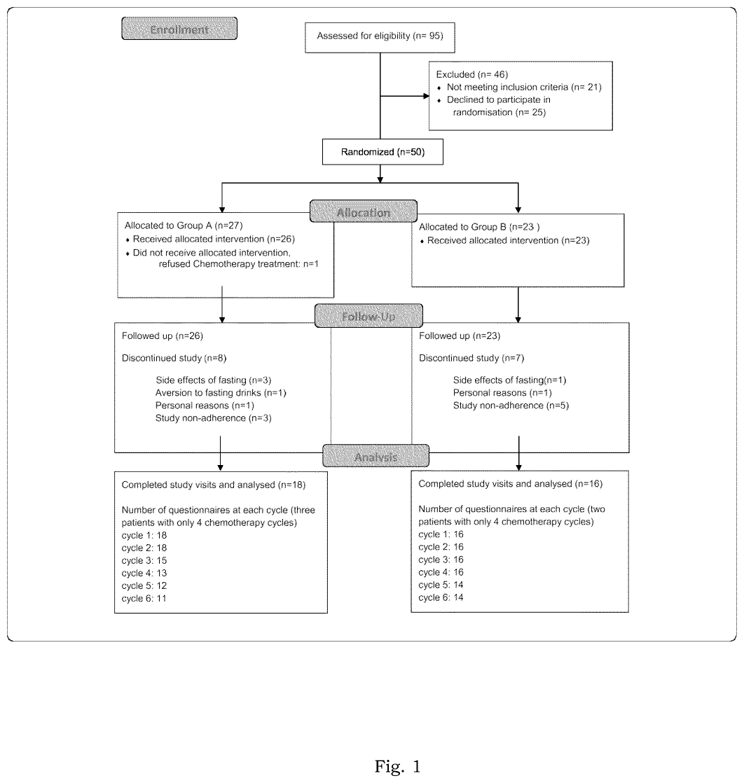

121 patients were screened, 50 patients fulfilled the inclusion criteria and were recruited to the study. The patient flow chart is given in FIG. 1. One patient cancelled the complete chemotherapy treatments initially. Although FMD was generally well tolerated, four patients withdrew from FMD after having experienced minor side effects of FMD like headache (two cases), hyperventilation during first chemotherapy (one case) and general subjective weakness (one case). One patient reported an aversion to fasting nutrients. Thus, in total 5 patients (10%) dropped out related to the fasting intervention. Two patients withdrew from the study because of personal reasons (family problems), each in the diet phase. Eight subjects could not be assessed for the follow-ups as a result of non-adherence with the study because of time restrictions and complete unwillingness to fill out further study documents (three in the FMD phases, five in the normocaloric diet phases). These drop-outs were not related to any adverse effects of the study interventions as assessed by telephone interviews. A total of 34 patients with primary BC (n=29), advanced BC (n=1) and OC (n=4) were analyzed.

### Interventions

All patients received standard oncological care as determined by their individual requirements. Patients in both groups were advised to maintain their regular physical activity and to abstain from other new integrative or supportive treatments during the study period.

### Chemotherapeutic Drugs and Standard Therapy

Chemotherapeutic drugs used in this study were taxanes (docetaxel (D), paclitaxel (T)), platinum agents (carboplatin (P), cyclophosphamide (C), anthracyclines (epirubicin (E), doxorubicin (A), methotrexatate (M), fluorouracil (F), the IgG1 antibody bevacizumab (Avastin) and for patients with HER2/neu overexpression pertuzumab or trastuzumab. These were given in various standard combinations according to guideline-based treatment protocols. For patients with breast cancer the following regimens: EC, FEC-D; FEC-D+trastuzumab, AC-T,EC-T,TAC and D+pertuzumab+trastuzumab were used. For patients with ovarian cancer P mono, P+T, EC-T+P and P+T+bevacizumab were used. Standard antiemetics and medication to prevent hypersensitivity reactions were administered, including dexamethasone and 5HT3 inhibitors.

### FMD Intervention

All patients received individual dietary counselling according to their individual needs in order to correctly perform the FMD program and the normocaloric diet. The FMD program was adapted to the established approach of modified fasting used in our hospital for many years and evaluated in studies in patients with rheumatic diseases and chronic pain [16]. The FMD period started 36 h before chemotherapy and ended 24 h after the end of the chemotherapy resulting in a total FMD period of 60 h. During the FMD period subjects received unrestricted amounts of water, herbal tea, and a wide variety of vegetable juices and small standardized quantities of light vegetable broths aimed at maximizing compliance and minimizing food aversion (the association of specific food with the side effects caused by the cancer therapy). The total daily energy intake was approximately 350 kcal. Compliance with the FMD regimen was assessed by telephone calls and personal interviews at the end of the chemotherapy cycles.

### Standard Nutrition Period

All patients were advised to follow a normocaloric Mediterranean diet throughout this study phase including the chemotherapy days.

### Randomization

Patients were randomly allocated to treatment groups (block-randomization with randomly varying block sizes (SAS/Base® statistical software (SAS/Base® statistical software)). For each patient sealed, sequentially numbered envelopes containing treatment assignments were prepared. Allocation of treatment was not blinded.

### Inventories for Outcome Assessment

For measuring health-related QOL, the functional assessment of chronic illness therapy (FACIT©) measurement system was used. The Functional Assessment of Cancer Therapy-General (FACT-G©) forms the generic core questionnaire of all FACIT© scales. The FACIT© scales are constructed to complement the FACT-G© scale by addressing relevant disease-, treatment-, or condition-related issues not already covered in the general questionnaire. The Trial Outcome Index (TOI) is a measure of physical aspects of QOL. It is the sum of the FACT-G subscales of physical well-being (PWB), functional well-being (FWB), and any FACIT© disease-, treatment-, or condition-specific scale. For additional concerns, the FACIT-F©, a 13-item questionnaire that assesses self-reported fatigue and its impact upon daily activities, was used. Altogether, 8 different scales and subscale scores were obtained: the subscales PWB, EWB, SWB, FWB as compounds of the FACT-G© scale (27 items); the additional subscale FACIT-F© (13 items); the TOI-FACIT-F© (27 items) and the total FACIT-F© scale as union of the FACT-G© and FACIT-F© scales (40 items).

### Statistical Analysis

This randomized cross-over pilot study aimed to give first insight into QOL and tolerability to chemotherapy in breast or ovarian cancer patients with FMD versus normocaloric diet during chemotherapy. As a pilot study it aimed to enable the design of a further confirmatory trial that is anticipated.

All FACIT© scales were scored with a higher score indicating better well-being. Here, response scores were reversed on negatively phrased questions, then the item responses were added. The scores were obtained in accordance with the formula that had been previously established by the FACIT© system. In cases where individual questions were skipped, scores were prorated using the average of the other answers in the subscale (prorated subscale score=[sum of the item scores]*[N of items in subscale]/[N of items answered]) as long as more than 50% of the items were answered (minimum of 4 items for the subscales). The FACT-G score was considered appropriate as long as at least 22 of 27 FACT-G items were completed (≥30%). Inter-subscale correlations were computed using Pearson correlation, and the reliability of the internal consistency for all scales were assessed by computing Cronbach's alpha. When Cronbach's alpha exceeded 0.90, the scale was considered to have sufficient precision for individual classification or diagnosis. MIDs of FMD and non-fasting groups were used to find clinically meaningful improvements in symptoms and to aid the interpretation of group differences and changes in QOL over time. A MID for scores of scales is defined by the “smallest change in a score in the domain of interest that patients perceive as important, either beneficial or harmful, and that would lead the clinician to consider a change in the patient's management”[17]. MID values over 3-7 (mean 5) for FACT-G© and over 3-4 for Fatigue subscale and 6 for Total FACT-F were considered significant.

The sample size was calculated for the group A starting with STF assuming an equivalence margin of 5 i.e. the MID of FACT-G, a true difference of 7 and a standard deviation of 3. A sample size of 16 achieves a power 82% with significance level of 0.05. Assuming the same condition for the group B, 32 patients were obtained as minimum.

Continuous variables with a normal distribution were expressed as mean value and standard deviation. Normality was tested with the Shapiro-Wilks test. Statistical comparison of baseline characteristics and outcomes was performed using the x2 test with Yates correction or the Fisher exact test, when appropriate, for categorical variables. For continuous variables, used the two-tailed Student's t-test respectively the independent-samples Mann-Whitney U test was used in order to test the null hypothesis that the distribution of a variable is the same across the groups A and B.

Calculations were performed with NCSS (Version 10), R (version 3.1.0) and IBM SPSS Statistics for Windows (Version 22.0. Armonk, N.Y.: IBM Corp).

### Results

The median age of the 34 patients was 51 y (range 28-69 y). 18 patients started with FMD during the first half of chemotherapy cycles (group A) whereas the other 16 patients (group B) started with normal diet (table 1).

TABLE 1

| | | | | Group A | | Group B | | |
|---|---|---|---|---|---|---|---|---|
| Basic characteristics | | Total 34 | | 18 (52.9%) | | 16 (47.1%) | | Significance |
| Age at diagnosis | | mean: 51.6 | | mean: 49.8 | | mean: 53.6 | | 0.195 |
| | | (SD 8.4) | | (SD 9.1) | | (SD 7.3) | | |
| | | (median: 51) | | (median: 51) | | (median: 52) | | |
| | | Range: 28-69 | | (Range: 28-63 | | Range: 44-69 | | |
| Menopausal status | Premenopausal | 24 | | 13 | (72.2) | 11 | (68.7) | 0.824 |
| Tumor entity | Postmenopausal | 10 | | 5 | (27.8) | 5 | (31.3) | |
| | primary breast cancer | 25 | (73.5) | 12 | (66.7) | 13 | (81.3) | 0.691 |
| | advanced breast cancer | 5 | (14.7) | 3 | (16.7) | 2 | (12.5) | |
| | ovarian cancer | 3 | (8.8) | 2 | (11.1) | 1 | (6.3) | |
| | advanced ovarian cancer | 1 | (2.9) | 1 | (5.6) | 0 | (0.0) | |
| T-categories | T0 | 1 | (2.9) | 0 | (0.0) | 9 | (56.3) | 0.323 |
| | T1 | 14 | (41.2) | 7 | (38.9) | 7 | (43.8) | |
| | T2 | 13 | (38.2) | 9 | (50.0) | 4 | (25.0) | |
| | T3 | 6 | (17.6) | 2 | (11.1) | 4 | (25.0) | |
| Nodal status | pNx | 1 | (2.9) | 1 | (5.6) | 0 | (0.0) | 0.434 |
| | pN0 | 16 | (47.1) | 7 | (38.9) | 9 | (56.3) | |
| | pN1 | 17 | (50.0) | 10 | (55.6) | 7 | (43.8) | |
| Grading | G1 | 1 | (2.9) | 1 | (5.6) | 0 | (0.0) | 0.298 |
| | G2 | 17 | (50.0) | 7 | (38.9) | 10 | (62.5) | |
| | G3 | 16 | (47.1) | 10 | (55.6) | 6 | (37.5) | |
| Breast cancer | Luminal A | 3 | (10.0) | 3 | (20.0) | 0 | (0.0) | 0.098 |
| subtypes (n = 30) | Luminal B/HER2− | 11 | (36.7) | 4 | (26.7) | 7 | (46.7) | |
| | Luminal B/HER2+ | 4 | (13.3) | 2 | (13.3) | 2 | (13.3) | |
| | triple negative | 9 | (30.0) | 6 | (40.0) | 3 | (20.0) | |
| | HER2 overexpressing | 3 | (10.0) | 0 | (0.0) | | | |
| Therapy (breast | CT | 25 | (83.3) | 13 | (86.7) | 12 | (80.0) | 0.624 |
| | CT + Anti-HER2 | 5 | (16.7) | 2 | (13.3) | 3 | (20.0) | |
| Therapy (ovarian | CT | 3 | (75.0) | 3 | (100.0) | 0 | (0.0) | 0.046 |
| | CT + Anti-HER2 + Anti-VEGF | 1 | (25.0) | 0 | (0.0) | 1 | (100.0) | |
| Body Mass Index | normal (BMI ≤25) | 19 | (55.9) | 9 | (50.0) | 10 | (62.5) | 0.364 |
| | overweight (BMI: 25-30) | 13 | (38.2) | 7 | (38.9) | 6 | (37.5) | |
| | obese (BMI >30) | 2 | (5.9) | 2 | (11.1) | 0 | (0.0) | |

Legend: Baseline characteristics for both groups.
Group A: FMD for the first half of cycles of chemotherapy and eating a normocaloric diet for the second half of cycles;
Group B: Eating a normocaloric diet for the first half of cycles of chemotherapy and FMD for the second half of cycles

The FMD was safe and all reported side effects were of low grade and at a level that did not interfere with daily activities. Minor adverse effects during all cycles, and mainly during the first FMD cycle, included headache (5×), hunger (5×), slight nausea after intake of broth or juices (11×) and one orthostatic reaction. There was one wound infection, unrelated to FMD. Regarding tolerability of chemotherapy there were no common toxicity criteria grade III/IV adverse events documented.

### Changes in Body Weight/Body Mass Index

Weight gain is a common problem for breast/ovarian cancer patients treated with chemotherapy and in particular with anthracyclines+taxanes [18]. In this trial there were no significant (p>0.3) changes in weight. Mean body weight (BMI) of the patients in group A was 73 kg (26.1) at the beginning and 72.3 kg (25.8) at the end of the trial. For group B the corresponding values are 67.9 kg (23.7) and 68.5 kg (24.2).

Throughout all chemotherapy cycles of the 34 patients the Applicants documented 102 cycles fasted (FMD) and 74 cycles on normocaloric diet. The numerical difference between fasted (FMD) and non-fasted cycles is a result of 5 patients who did not want to switch to normocaloric diet after FMD for the first 3 cycles. Compliance with the FMD protocol, as checked by diaries, telephone calls and interviews appeared good.

### Baseline

The means of the various scales and subscales were worse for patients with ovarian cancer compared to patients with breast cancer at baseline (day 0, cycle 1) (OC vs BC: FACT-G 66.8 (±21.7) vs 80.5 (±15.3); FACIT-F 33.5 (±10.3) vs 39.7 (±10.2); Total FACIT-F 100.3 (±31.8) vs 120.2 (±21.7)). The internal consistencies were excellent for the various questionnaires: (Cronbach's alpha: a>0.9). FACT-G (a=0.91), FACIT-F (a=0.96), TOI FACIT-F (a=0.97) and total FACIT-F (a=0.96). There were statistically no significant differences in FACT-G respectively its subscales between both groups at baseline except for Social/Family Well-Being p=0.042).

### Chemotherapy Courses

A 2×2 crossover design AB|BA (A=FMD; B=normocaloric diet) with cross-over at cycle 4 was used. Five patients had a total of only four cycles of chemotherapy, so the first two cycles were aligned to c1 and c2 and the second two cycles to c4 and c5.

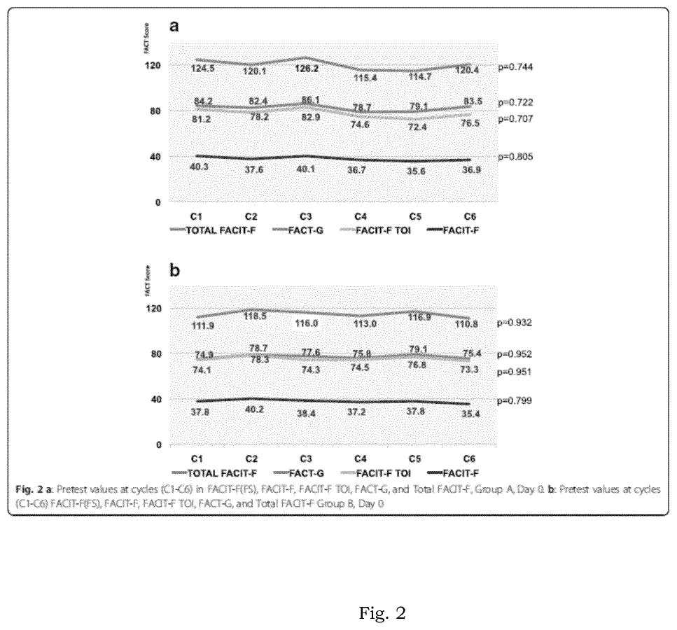

The mean values of all scales at day 0 (before chemotherapy) were not significantly different for group A and B across the 6 cycles, i.e. the patients recovered within three weeks from each cycle of chemotherapy with respect to QOL (FIG. 2a/b) Hence, there seemed to be no carryover effects from FMD respectively normocaloric diet during the first three cycles to the second period.

Change of QOL after Chemotherapy

Within group A a statistically and clinically significant beneficial effect of the FMD during chemotherapy (cycles c1-c3) versus normocaloric diet (cycles c4-c6) on QOL and fatigue was found. In group B fasted (FMD) cycles (c4-c6) were not associated with a significant reduction of chemotherapy-induced QOL and fatigue compared to regular diet (c1-c3) (table 2).

TABLE 2
Descriptives

| | | | | | 95% Confidence | | | |
|---|---|---|---|---|---|---|---|---|
| | | | | | Interval for Mean | | | |
| | | | | Std. | Lower | Upper | Mean | Statistical |
| Group | Cycle | N | Mean | Deviation | Bound | Bound | difference | significance |
| FACT-G Group A | first half of a 4-6 cycle | 52 | 71.1 | 18.7 | 65.9 | 76.3 | 9.2 | 0.041 |
| | second half of a 4-6 cycle | 27 | 61.9 | 18.5 | 54.6 | 69.2 | | |
| FACT-G Group B | first half of a 4-6 cycle | 46 | 76.0 | 17.2 | 70.9 | 81.1 | 2 | 0.576 |
| | second half of a 4-6 cycle | 47 | 74.0 | 17.6 | 68.8 | 79.2 | | |
| FACIT-F (FS) Group A | first half of a 4-6 cycle | 52 | 33.9 | 13.4 | 30.2 | 37.6 | 9.1 | 0.006 |
| | second half of a 4-6 cycle | 27 | 24.8 | 13.7 | 19.4 | 30.2 | | |
| FACIT-F (FS) Group B | first half of a 4-6 cycle | 46 | 33.4 | 14.0 | 29.3 | 37.6 | 1.8 | 0.521 |
| | second half of a 4-6 cycle | 47 | 31.7 | 12.6 | 28.0 | 35.4 | | |
| FACIT-F TOI Group A | first half of a 4-6 cycle | 52 | 66 | 25 | 59 | 72.9 | 16.2 | 0.009 |
| | second half of a 4-6 cycle | 27 | 49.8 | 26.4 | 39.4 | 60.3 | | |
| FACIT-F TOI Group B | first half of a 4-6 cycle | 46 | 68.0 | 25.2 | 60.5 | 75.5 | 4.2 | 0.41 |
| | second half of a 4-6 cycle | 47 | 63.8 | 23.9 | 56.8 | 70.8 | | |
| FACIT-F Total Group A | first half of a 4-6 cycle | 52 | 105 | 30.4 | 96.6 | 113.5 | 18.3 | 0.013 |
| | second half of a 4-6 cycle | 27 | 105 | 30.5 | 74.6 | 98.8 | | |
| FACIT-F Total Group B | first half of a 4-6 cycle | 46 | 109.5 | 29.8 | 100.6 | 118.3 | 4.2 | 0.531 |
| | second half of a 4-6 cycle | 47 | 105.7 | 28.3 | 97.3 | 114.0 | | |

Mean and standard deviations for outcome parameters with mean group differences and 95% CI for FACT-G, FACIT-F, TOI FACIT-F, and Total FACIT-F between cycles with FMD and regular diet of group A and B at day 8 after chemotherapy

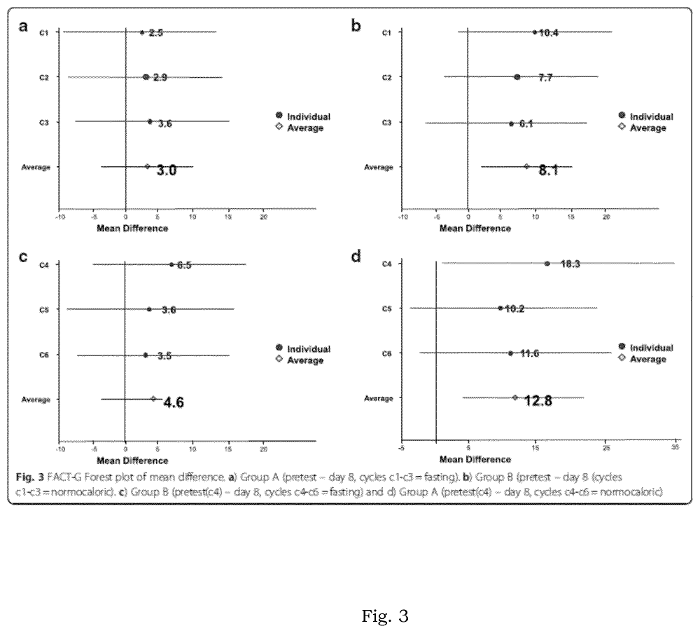

When analysing changes of QOL within the fasted (FMD) or non-fasted periods by applying minimally important differences (MID for FACT-G=5), for patients in group A within the first three FMD cycles a decrease of FACT-G (mean=3.0) was found that was less than the MID. Thus, patients under FMD did not perceive the change between FACT-G at day 0 and day 8 after chemotherapy as important. In contrast, the change of QOL for patients of group A across the normocaloric diet cycles c4-c6 was greater than the MID (mean 12.8), these patients perceived the chemotherapy-induced reduction of QOL as important [FIG. 3 a,d].

Accordingly, the mean differences (day 0-day 8) of TOI FACIT-F of group A was 10.5 for first three cycles and 25.1 for cycles 4-6. Patients on FMD within the first three cycles had a clinically significantly lower difference TOI FACIT-F compared to the same patients during cycles 4-6 on a regular diet.

The mean differences TOI FACIT-F of group B was 13.1 for first three cycles and 10.8 for cycles 4-6. Patients on FMD within the first three cycles had a clinically significantly lower difference TOI FACIT-F compared to the same patients during cycles 4-6 on a regular diet. There was no significant difference between the mean differences of TOI FACIT-F for group A and group B during the FMD cycles (10.5 vs 10.8).

In group B no statistically significant differences between the first 3 cycles and the second three cycles was found. However, when analysing changes of QOL within the fasted (FMD) or non-fasted periods by applying MID, an average of mean difference of FACT-G for the cycles with FMD of 4.6 and with normocaloric diet of 8.1 [FIG. 3 b, c] was found. Thus, again patients with normocaloric diet had a mean difference above the MID in that phase whereas with FMD the mean difference was 4.6, i.e. below the MID. FIG. 3a-d summarizes the forest plots of the chemotherapy-induced changes of FACT-G in each of the two dietary phases in both groups.

In general, FMD was very well accepted by the patients. At the final consultation the majority of patients reported better tolerance to chemotherapy with FMD, the patient general assessment of the effectiveness of FMD revealed “good” or “very good” for 28 patients, “moderate” in 5 patient s and “no effect” in 1 patient. 31 patients declared that they would fast again during chemotherapy, 3 patients declared that they would not like to fast again during chemotherapy.

In conclusion, FMD with a period of 60 h did not induce weight loss and was associated with only minor adverse effects that were rated as not meaningful by the patients and did not interfere with daily activities.

In general, FMD led to a better tolerance to chemotherapy with less compromised QOL and reduced fatigue within the 8 days after chemotherapy. As QOL is an increasingly appreciated treatment outcome, the present results appear to be of clinical relevance.

No evidence of malnutrition was found.

### REFERENCES

1. Lee C, Raffaghello L, Brandhorst S, Safdie F M, Bianchi G, Martin-Montalvo A, Pistoia V, Wei M, Hwang S, Merlino A et al: Fasting cycles retard growth of tumors and sensitize a range of cancer cell types to chemotherapy. Science translational medicine 2012, 4(124):124ra127.
2. Longo V D, Mattson M P: Fasting: molecular mechanisms and clinical applications. Cell metabolism 2014, 19(2):181-192.
3. Tannenbaum A, Silverstone H: The influence of the degree of caloric restriction on the formation of skin tumors and hepatomas in mice. Cancer research 1949, 9(12):724-727.
4. Michalsen A, Schneider S, Rodenbeck A, Lüdtke R, Huether G, Dobos G J: The Short-term Effects of Fasting on the Neuroendocrine System in Patients with Chronic Pain Syndromes. Nutritional Neuroscience 2003, 6(1):11-18.
5. Michalsen A, Li C, Kaiser K, Lüdtke R, Meier L, Stange R, Kessler C: In-Patient Treatment of Fibromyalgia: A Controlled Nonrandomized Comparison of Conventional Medicine versus Integrative Medicine including Fasting Therapy. Evidence-based Complementary and Alternative Medicine: eCAM 2013, 2013:908610.
6. Choi I Y, Piccio L, Childress P, Bollman B, Ghosh A, Brandhorst S, Suarez J, Michalsen A, Cross A H, Morgan T E et al: Diet mimicking fasting promotes regeneration and reduces autoimmunity and multiple sclerosis symptoms. Cell reports 2016, 15(10):2136-2146.
7. Walford R L, Harris S B, Weindruch R: Dietary restriction and aging: historical phases, mechanisms and current directions. The Journal of nutrition 1987, 117(10):1650-1654.
8. Safdie F M, Dorff T, Quinn D, Fontana L, Wei M, Lee C, Cohen P, Longo V D: Fasting and cancer treatment in humans: A case series report. Aging 2009, 1(12):988-1007.
9. Fontana L, Partridge L, Longo V D: Extending healthy life span—from yeast to humans. Science 2010, 328(5976):321-326.
10. Colman R J, Anderson R M, Johnson S C, Kastman E K, Kosmatka K J, Beasley T M, Allison D B, Cruzen C, Simmons H A, Kemnitz J W et al: Caloric restriction delays disease onset and mortality in rhesus monkeys. Science 2009, 325(5937):201-204.
11. Lee C, Longo V D: Fasting vs dietary restriction in cellular protection and cancer treatment: from model organisms to patients. Oncogene 2011, 30(30):3305-3316.
12. Raffaghello L, Lee C, Safdie F M, Wei M, Madia F, Bianchi G, Longo V D: Starvation-dependent differential stress resistance protects normal but not cancer cells against high-dose chemotherapy. Proceedings of the National Academy of Sciences of the United States of America 2008, 105(24):8215-8220.
13. Lee C, Safdie F M, Raffaghello L, Wei M, Madia F, Parrella E, Hwang D, Cohen P, Bianchi G, Longo V D: Reduced levels of IGF-I mediate differential protection of normal and cancer cells in response to fasting and improve chemotherapeutic index. Cancer research 2010, 70(4):1564-1572.
14. Michalsen A, Kuhlmann M K, Lüdtke R, Backer M, Langhorst J, Dobos G J: Prolonged fasting in patients with chronic pain syndromes leads to late mood-enhancement not related to weight loss and fasting-induced leptin depletion. Nutritional Neuroscience 2006, 9(5-6):195-200.
15. Boschmann M, Michalsen A: Fasting Therapy—Old and New Perspectives. Forschende Komplementärmedizin/Research in Complementary Medicine 2013, 20(6):410-411.
16. Michalsen A, Li C: Fasting therapy for treating and preventing disease—current state of evidence. Forschende Komplementarmedizin (2006) 2013, 20(6):444-453.
17. Schunemann H J, Akl E A, Guyatt G H: Interpreting the results of patient reported outcome measures in clinical trials: the clinician's perspective. Health and quality of life outcomes 2006, 4:62.
18. Winkels R M, Beijer S, van Lieshout R, van Barneveld D, Hofstede J, Kuiper J, Vreugdenhil A, van Warmerdam L J C, Schep G, Blaisse R et al: Changes in body weight during various types of chemotherapy in breast cancer patients. e-SPEN Journal, 9(1):e39-e44.
19. de Groot S, Vreeswijk M P, Welters M J, Gravesteijn G, Boei J J, Jochems A, Houtsma D, Putter H, van der Hoeven J J, Nortier J W et al: The effects of short-term fasting on tolerance to (neo) adjuvant chemotherapy in HER2-negative breast cancer patients: a randomized pilot study. BMC cancer 2015, 15:652.
20. Dorff T B, Groshen S, Garcia A, Shah M, Tsao-Wei D, Pham H, Cheng C W, Brandhorst S, Cohen P, Wei M et al: Safety and feasibility of fasting in combination with platinum-based chemotherapy. BMC cancer 2016, 16(1):360.

1-44. (canceled)

45. A method of increasing the Quality-Of-Life (QOL) and reducing the fatigue of a human patient with cancer being subjected to therapy cycles with an anti-cancer agent, which method comprises subjecting said patient to a plurality of therapy cycles with said anti-cancer agent and subjecting said patient to a fasting mimicking diet (FMD) for a period of 30-42 hours before each therapy cycle and 18-30 hours after each therapy cycle.

46. The method according to claim 45, wherein said period of FMD is of 33-39 hours before each therapy cycle and of 21-27 hours after each therapy cycle.

47. The method according to claim 46, wherein said period of FMD is of 36 hours before each therapy cycle and of 24 hours after each therapy cycle.

48. The method according to claim 45, wherein said plurality of therapy cycles consists of 4-6 therapy cycles.

49. The method according to claim 45, wherein said anti-cancer agent is a chemotherapeutic agent and said therapy cycles are chemotherapy cycles.

50. The method according to claim 49, wherein said chemotherapeutic agent is selected from a group consisting of DNA alkylating agents, taxanes, platinum agents, cyclophosphamide, anthracyclines, topoisomerase inhibitors, kinase inhibitors, nucleotide analogs, retinoids, peptide antibiotic, Vinca alkaloids, epothilones, histone deacetylase inhibitors, antibodies.

51. The method according to claim 50, wherein said chemotherapeutic agent is selected from the group consisting of docetaxel, paclitaxel, carboplatin, cyclophosphamide, epirubicin, doxorubicin, methotrexate, fluorouracil, bevacizumab, pertuzumab and trastuzumab.

52. The method according to claim 45, wherein said FMD is a food composition providing a daily caloric intake reduced by 50-90%.

53. The method according to claim 45, wherein said FMD is a food composition providing a daily caloric intake reduced by 85-90%.

54. The method according to claim 52, wherein said reduced caloric intake corresponds to a caloric intake of 100-350 kcal/day.

55. The method according to claim 45, wherein said cancer is gynecological cancer.

56. The method according to claim 55, wherein said gynecological cancer is breast cancer or ovarian cancer.

57. The method according to claim 45, wherein said FMD is a food composition with reduced caloric, sugar and/or protein content but containing all necessary micronutrients to prevent malnutrition.

58. The method according to claim 57, wherein said FMD is a food composition that comprises proteins in such an amount that provides less than 15% of the total calories provided by the FMD, and that comprises sugars in such an amount that provides less than 15% of the total calories provided by the FMD.

59. The method according to claim 58, wherein said FMD is a food composition that comprises at least 60% calories from fatty acids, 2-5% calories from glycerol and up to 5% calories from plant-based proteins and a maximum of 35% calories from carbohydrates.

60. An improved method of treating a human patient with cancer, comprising a step of subjecting said patient to a plurality of therapy cycles with an anti-cancer agent, the improvement consisting in subjecting said patient to a fasting mimicking diet (FMD) for a period of 30-42 hours before each therapy cycle and 18-30 hours after each therapy cycle, thus obtaining an increase of the patient's Quality-Of-Life (QOL) and a fatigue reduction.

61. The method according to claim 60, wherein said period of FMD is of 33-39 hours before each therapy cycle, and of 21-27 hours after each therapy cycle.

62. The method according to claim 60, wherein said period of FMD is of 36 hours before each therapy cycle, and of 24 hours after each therapy cycle.

63. The method according to claim 60, wherein said plurality of therapy cycles consists of 4-6 therapy cycles.

64. The method according to claim 60, wherein said anti-cancer agent is a chemotherapeutic agent and said therapy cycles are chemotherapy cycles.

65. The method according to claim 64, wherein said chemotherapeutic agent is selected from a group consisting of DNA alkylating agents, taxanes, platinum agents, cyclophosphamide, anthracyclines, topoisomerase inhibitors, kinase inhibitors, nucleotide analogs, retinoids, peptide antibiotic, Vinca alkaloids, epothilones, histone deacetylase inhibitors, antibodies.

66. The method according to claim 65, wherein said FMD is a food composition providing a daily caloric intake reduced by 50-90%.

67. The method according to claim 65, wherein said FMD is a food composition providing a caloric intake of 100-350 kcal/day.

68. The method according to claim 65, wherein said cancer is gynecological cancer.

69. The method according to claim 68, wherein said gynecological cancer is breast cancer or ovarian cancer.
